(19) United States
(12) Patent Application Publication
(10) Pub. No.: US 2016/0222135 A1
(43) Pub. Date: Aug. 4, 2016

(54) SYSTEM FOR AND METHOD OF SEPARATING PURE STARCH FROM GRAINS FOR ALCOHOL PRODUCTION USING A DRY MILL PROCESS

(71) Applicant: Lee Tech LLC., San Jose, CA (US)

(72) Inventor: Chie Ying Lee, San Jose, CA (US)

(21) Appl. No.: 15/008,319

(22) Filed: Jan. 27, 2016

Related U.S. Application Data
(60) Provisional application No. 62/109,424, filed on Jan. 29, 2015.

(51) Int. Cl. C08B 30/04; C08B 30/02; C12P 7/16

(52) U.S. Cl. CPC C08B 30/044; C12P 7/16; C08B 30/02

(57) ABSTRACT

Methods of and systems for recovering starch before fermentation in a dry mill process and/or a wet mill process. The starch is able to be further purified. The starch can be used as a feedstock for biotech uses, such as, making one or more types of butanols. The method is able to recover starch from floury and horny endosperm. In some embodiments, the method includes liquefying milled corn and separating, purifying and recovering the starch in both floury and horny endosperm before fermentation. In some embodiments, the method includes subjecting the milled corn flour in a caustic condition and next recovering the starch before fermentation. In some embodiments, the method includes soaking/steeping the corn, grinding the corn and separating the germ from the starch in a wet mill similar condition before fermentation.

### RELATED APPLICATIONS

This Patent Application claims priority under 35 U.S.C. 119 (e) of the co-pending U.S. Provisional Application Ser. No. 62/109,424, filed Jan. 29, 2015, and entitled “A SYSTEM FOR AND METHOD OF SEPARATING PURE STARCH FROM GRAINS FOR ALCOHOL PRODUCTION USING A DRY MILL PROCESS.” This application incorporates U.S. Provisional Application Ser. No. 62/109,424 in its entirety by reference.

### FIELD OF THE INVENTION

The present invention relates to a dry mill plant. Specifically, the present invention relates to systems for and methods of separating pure raw starch and/or liquefied starch.

### BACKGROUND OF THE INVENTION

FIG. 1 is a typical wet mill process for alcohol production. FIG. 2 is a typical dry mill process with a back end oil recovery system. FIG. 3 is a typical dry mill process with a back end oil and protein recovery system. FIG. 4 is a typical dry mill process with a front end grind milling and front end oil recovery system.

The conventional methods of producing various types of alcohols from grains generally follow similar procedures depending on whether the grain grinding process is operated wet or dry. Wet mill corn processing plants convert corn grain into several different co-products, such as germ (for oil extraction), gluten feed (high fiber animal feed), gluten meal (high protein animal feed), and starch-based products (such as, ethanol, high fructose corn syrup, or food) and industrial starch (such as, bio tech processes feedstock).

Dry grind ethanol plants convert corn into two products, namely ethanol and distiller's grains with soluble. If sold as wet animal feed, distiller's wet grains with soluble is referred to as DWGS. If dried for animal feed, distiller's dried grains with soluble is referred to as DDGS. In the standard dry grind ethanol process, one bushel of corn yields approximately 8.2 kg (approximately 17 lbs.) of DDGS in addition to the approximately 10.3 liters (approximately 2.75 gal) of ethanol. These co-products provide a critical secondary revenue stream that offsets a portion of the overall ethanol production costs. DDGS is sold as a low value animal feed even though that the DDGS contains 11% oil and 30% protein, dry matter basis (DMB). Some plants have started to modify the typical process to separate oil and protein from DDGS.

With respect to the wet mill process, FIG. 1 shows a flow diagram of a typical wet mill ethanol production process 10. The process 10 begins with a steeping step 11, in which corn is generally soaked for about 24 to 48 hours in a solution of water and sulfur dioxide in order to soften the kernels for grinding, leach soluble components into the steep water, and loosen the protein matrix with the endosperm. Corn kernels contain mainly starch, fiber, protein, and oil. The steeped corn (after the Steeping step 11) with about 50% DS (dry solids) is then fed to a determination milling step (first grinding) 12 at a grind mill, in which the corn is ground in a manner that tears open the kernels and releases the germ so as to make a heavy density (8 to 9.5 Be) slurry of the ground components- primarily a starch slurry. This is followed by a germ separation step 13 by flotation and the use of a hydrocyclone to separate the germ from the rest of the slurry. The germ is the part of the kernel that contains the majority of the oil in a corn kernel. The separated germ stream (separated out as a germ byproduct), which contains some portion of the starch, protein, and fiber, goes to the germ washing process to remove excess starch and protein, and then to a dryer to produce about 2.5 to 3 lb. (dry basis) of germ per bushel of corn. The dry germ has about 50% oil content on a dry basis.

The remaining slurry at the step 13, which is now devoid of germ, but containing fiber, corn gluten (i.e., protein), and starch, is then subjected to a fine grinding step (second grinding) 14 at a fine grind mill. The fine grind produces near total disruption of endosperm and release of endosperm components, namely gluten and starch, from the fiber. The step 14 is followed by a fiber separation step 15 where the slurry is passed through a series of screens in order to separate the fiber from starch and gluten, and to wash the fiber, such that the fiber is clean and free of excessive gluten and excessive starch. The fiber separation stage 15 typically employs static pressure screens or rotating paddles mounted in a cylindrical screen (Paddle Screens).

Even after washing, the fiber from a typical wet grind mill contains 15 to 20% starch. This starch can be sold with the fiber as an animal feed. The remaining slurry of the step 15, which is now devoid of fiber, is subjected to a gluten separation step 16 in which centrifugation separates starch from the gluten. The gluten stream goes to step 16A in a vacuum filter followed by a drying step at a dryer to produce gluten (protein) meal.

For alcohol production, the starch from the starch gluten separation step 16 normally goes through jet cooker to start the process of converting the starch to sugar. Jet cooking refers to a cooking process that is performed at elevated temperatures and pressures. The elevated temperatures and pressures can vary widely. Typically, jet cooking occurs at a temperature about 120 to 150° C. (about 248 to 302° F.) and a pressure about 8.4 to 10.5 kg/cm² (about 120 to 150 lbs/in²), although the temperature can be as low as about 104 to 107° C. (about 220 to 225° F.) when a pressure of about 8.4 kg/cm² (about 120 lbs/in^{t}) is used.

The starch from the starch gluten separation step 16 is followed by a liquefaction and saccharification step 17, a fermentation step 18, a yeast recycling (not shown), and a distillation/dehydration step 19. Liquefaction occurs as the mixture, or “mash” is held at 90 to 95° C. allowing the alpha-amylase to hydrolyze the gelatinized starch into maltodextrins and oligosaccharides (chains of glucose sugar molecules), which produce a liquefied mash or slurry.

In the saccharification step 17, the liquefied mash is cooled to about 60° C. and a commercial enzyme known as gluco-amylase is added. The gluco-amylase hydrolyzes the maltodextrins and short-chained oligosaccharides into single glucose sugar molecules to produce a saccharified mash. In the fermentation step 18, yeast (most commonly Saccharomyces cerevisiae) is added to metabolize the glucose sugars into ethanol and CO₂.

Upon completion, the fermented mash (“beer”) commonly contains about 15% to 18% ethanol (volume/volume basis). Subsequent to the fermentation step 18 is the distillation and dehydration step 19, in which the beer is pumped into distillation stripping column(s) where the beer is boiled to vaporize the ethanol. The ethanol vapor is condensed in the rectifier distillation column(s), and liquid alcohol (in this instance, ethanol) exits the distillation system at about 95% purity (190 proof). The 190 proof ethanol then goes through a molecular sieve dehydration column, which removes the remaining residual water from the ethanol, to yield a final product of essentially 100% ethanol (199.5 proof). This anhydrous ethanol is now ready to be used for motor fuel purposes. The solids and some liquid remaining after distillation go to an evaporation stage 20, where yeast can be recovered as a byproduct. Yeast can optionally be recycled back to the fermenter. In some instances, the CO₂ is recovered and sold as a commodity product.

Centrifugation step is a required step at the end of the wet mill ethanol production process 10 as the condensed steep liquor (CSL), germ, fiber, and gluten have already been removed in the previous separation steps 11a, 13, 15, and 16. The “stillage” produced after distillation and dehydration 19 in the wet mill process 10 is “syrup.”

The wet mill process 10 can produce a high quality starch product for conversion to alcohol, as well as separate streams of germ, fiber and protein, which can be sold as byproducts to generate additional revenue streams. However, the wet mill process is complicated and costly, requiring high capital investments as well as high-energy costs for operation.

Because the capital costs of wet grind mills can be so prohibitive, some alcohol plants prefer to use a simpler dry grind process. FIG. 2 is a flow diagram of a typical dry grind ethanol production process 200. As a general reference point, the dry grind ethanol process 200 can be divided into a front end and a back end. The part of the process 200 that occurs prior to distillation 24/fermentation 23 is considered the “front end”, and the part of the process 20 that occurs after distillation 24/fermentation 23 is considered the “back end.” The “front end” and “back end” distinction can be used throughout the entire specification.

The front end of the process 200 begins with a grinding step 21, in which dried whole corn kernels are passed through hammer mills 21 to be ground into corn meal or a fine powder. The screen openings in the hammer mills are typically about 7/64″, or about 2.78 mm, with the resulting particle distribution yielding a very wide spread, bell type curve particle size distribution, which includes particle sizes smaller than 45 micron and larger than 2 to 3 mm

After hammer mills 21, a jet cooking process is used at the liquefaction 22. The temperature is maintained between about 50° C. to 105° C. for approximately 30 minutes to four (4) hours, so as to convert the insoluble starch in the slurry to soluble starch. The stream after the liquefaction step 22 has about 30% dry solids (DS) content with all the components contained in the corn kernels, including sugars, protein, fiber, starch, germ, grit, and oil and salt, for example. There are generally three types of solids in the liquefaction stream: fiber, germ, and grit, with all three solids having about the same particle size distribution. The liquefaction step 22 is followed by a simultaneous saccharification and fermentation step 23. This simultaneous step is referred to in the industry as “Simultaneous Saccharification and Fermentation” (SSF).

In some commercial dry grind ethanol processes, saccharification and fermentation occur separately (not shown). Both separated saccharification followed by fermentation and SSF can take as long as about 50 to 72 hours. Fermentation converts the sugar to alcohol using a fermenter. Subsequent to the saccharification and fermentation step 23 is a distillation (and dehydration) step 24, which utilizes a still to recover the alcohol.

The back end of the process 200, which follows distillation 24, includes a fiber separation step 25, which involves centrifuging the “whole stillage” produced with the distillation step 24 to separate the insoluble solids (“wet cake”) from the liquid (“thin stillage”).

The “wet cake” includes fiber (per cap, tip cap, and fine fiber), grit, germ particle and some proteins. The liquid from the centrifuge contains about 6% to 8% DS, which contains mainly oil, germ, fine fiber, fine grit, protein, soluble solids from the fermenter and ash from corn. In some plants, the whole stillage with about 12 to 14% DS is fed to first stage evaporator that is concentrated to 15 to 25% DS before feeding to fiber separation step 25.

The thin stillage is split into two streams, about 30 to 40% flow recycles back (“back set”) to be mixed with corn flour in a slurry tank at the beginning of the liquefaction step 22. The rest of the flow (about 60 to 70% of total flow) then enters evaporators in an evaporation step 27 to boil away moisture, leaving a thick syrup that contains mainly soluble (dissolved) solids from fermentation (25% to 40% dry solids). The back set water is used as part of the cooking water in liquefaction step 22 to reduce the fresh water consumption as well as save evaporating energy and equipment costs.

The concentrated slurry is able to be subjected to an optional oil recovery step 26, where the slurry can be centrifuged to separate oil from the syrup. The oil can be sold as a separate high value product. The oil yield is normally about 0.4 lbs/Bu of corn with high free fatty acids content. This oil yield recovers only about ¼ of the oil in the corn. About one-half of the oil inside the corn kernel remains inside the germ after the distillation step 24, which cannot be separated in the typical dry grind process using centrifuges. The free fatty acids content which is created when the oil is held in the fermenter for approximately 50 hours reduces the value of the oil.

The (de-oil) centrifuge only removes less than 50% oil in syrup because the protein and oil make an emulsion, which cannot be separated. The adding of chemicals, such as emulsion breaking additives, can improve the separation efficient in some degrees, but the chemicals are costly and the DDGS product can be contaminated by the added chemicals. Providing heat or raising the feed temperature at or prior to the centrifuge to break the emulsion is another way, but excessive heating negatively affects the color and quality of DDGS. Adding an alcohol to break the emulsion also improves the separation and increases the oil yield, but it needs expensive explosion-proof equipment and costly ethanol recovery operations. All those improvements only increase the oil yield from an average of 0.4 lbs/Bu to about average 0.6 lbs/Bu even though the “free” oil in the whole stillage is about 1 lbs/Bu. The oil/protein forms emulsion during the whole dry mill process which is the main reason for having a low oil yield in the back end oil system.

As shown in the process 30 of FIG. 3, the front end process can be as simple as an existing dry mill process. The process changes its procedure at a step after fiber separation 25 at the back end process. This oil/protein separation step 28 is added between fiber separation step 25 and the evaporator step 27. The nozzle centrifuges, disc centrifuges, or decanters are normally used for this application. The thin stillage from fiber separation step 25 is fed to the oil/protein separation centrifuge step 28. The oil/protein emulsion is broken in a higher G force inside the centrifuge, typically a disc centrifuge is required for sufficiently high G force. The oil goes to a light phase (overflow) discharge and the protein goes to a heavy phase discharge (underflow), because of the density difference between oil (density 0.9 gram/nil) and protein (1.2 gram/nil). The light phase (overflow) then is fed to an evaporator step 27 to be concentrated to contain 25 to 40% of DS (forming a semi-concentrated syrup). Next, the semi-concentrated syrup is sent to the back end oil recovery system step 26 to recover oil in the back end. The light phase stream contains less protein, so it has decreased tendency to form oil/protein emulsion.

The oil yield with this system can be as high as 1 lb./Bu. The de-oiled syrup from back end oil recovery step 26 can further be concentrated in an evaporator to a much higher syrup concentration, as high as about 60% of DS. The de-oiled syrup with low protein can avoid fouling at the evaporator even with the substantially higher DS concentration. The underflow from oil/protein separation step 28 goes to a protein dewater step 32 for protein recovery. The separated protein cake from protein dewater step 32 with a content of less than about 3% oil is sent to a protein dryer step 33 to produce a high value protein meal, which has a protein content of about 45-50%. The liquid from the protein dewater step 32 is sent back to the front end as a back-set liquid that is used as part of cooking water in the liquefaction step 22.

All of the oil that is recovered from the back end oil recovery system has poor quality, dark color, and high fatty acid around (15 to 20%), because the oil is in the fermenter more than 50 hours and been held at elevated temperatures for many hours after the distillation process. The back end oil separation is also difficult to be carried out, because the oil and protein form a stable emulsion. Each step in the dry mill process generally is accompanied by centrifugal pump transfer which tends to create oil/protein and/or oil/starch emulsion.

In order to get good quality oil and avoid the formation of the oil/protein emulsion during the dry mill process, recovering oil in the front end before multiple centrifugal pump transfers is a good solution. The three phase decanters that are used to recover the oil from the liquefied starch stream at the liquidation step are tested, but because of the high viscosity in liquefied starch solution plus the majority of the oil is still in a germ form during early liquefaction, the oil yield is normally low to around 0.2 lbs/Bu. Nonetheless, the oil quality from front end recovery is much better than oil obtained from the back end having lighter color and between 5 to 9% free fatty acid.

An improved front end oil recovery system has been developed to improve the oil yield as well as to increase the yield of the alcohol. As shown in the process 40 of FIG. 4, the two stages liquid/solid separation steps 42 and 44 are followed by two stage dewater milling steps 43 and 45 in series respectively with counter current setup. In the counter current setup, a portion of cook water (such as from a step of solid/liquid separation at the step 49) is added to holding tank step 46 instead of adding to the slurry tank step 41.

In the process 40, the cook water is mixed with a cake from the second dewater milling step 45, then the mixture is fed to a third solid/liquid separation step 49 to recover liquid which is about 2 to 3 degree of Brix. The liquid from step 49 is then mixed with the cake from the first dewater milling step 43, then transferred to the holding tank 46 for about 2 to 4 hours residence time. The content in the holding tank 46 is then fed to a second solid liquid separation step 44 to separate the liquid and fine suspended material from the coarse suspended material solids. The liquid separated at step 44 has about 6 to 8 Brix, which is now used as part of the cook water to be mixed with corn flour from the hammer mill and roller mill step 21, to be sent to the slurry tank step 41. Using this counter current washing setup, the germ particles have about twice the contact time in the holding tank, step 46, as a traditional dry grind process. The leaching of the oil from the germ is enhanced by this longer contact time as well as much lower Brix (around 6 to 8 Brix instead of 25 to 30 Brix) solution of liquefied starch solution.

The germ that is soaked in a lower Brix environment and has double the holding time in the liquefaction step softens more completely, such that the germ can be broken up from the shell and more completely release the oil at the second dewater grind milling step 45. This counter current washing setup process 40 also gives middle size germ particle from the second stage dewater milling 45, which is recycled back to the first dewater mill stage to ensure that the softened germ particles is milled more completely to become a smaller and more homogeneously sized germ particle (such as smaller than 150 micron) to release more oil.

All grit/germ/fiber solid particles have a wide range of particle size range from less than 45 micron to as large 2 to 3 mm Softening the germ particles in a lower Brix solution coupled with a longer holding time, the germ is much softer and easier to be broken up than the more recalcitrant fibers. Accordingly, the dewatered milling method can break up more germ particles while preserving critical fiber length allowing more effective separation of the long fibers from the rest of the cook medium. However, each dewatered milling step can only reduce the germ particle size by about half of its original size at best. For example, the germ particle of an average size of 1,000 micron becomes about 600 micron, on average, after one dewatered milling step. For germ particles to effectively release oil, the germ particle size is preferred to be less than 150 micron. Therefore, normally at least two or three stages dewatered millings in series are needed to release maximum oil from the germ particles.

The counter current washing setup allows middle size germs after second dewater milling step 44 to be recycled back to first dewater milling step 42 for breaking the germ particles one more time. The screen size opening on first and second solid/liquid separation steps 42 and 44 is selected to give a desired degree of sizes and recycling the germ particle to the slurry tank.

At a step 41, corn flour from hammer mill from the step 21 mixes with liquid stream from second solid/liquid separation step 44 at the slurry tank with an optional use of a jet cooker. The slurry from step 41 goes to the first solid/liquid separation step 42, such that the liquid is separated from the solid. At the step 42, the liquid that contains oil and small solid grain particles (germ, protein, and fine fiber) forms a liquefied starch solution, which is sent to the front end oil recovery step 47.

At the step 41, the de-water solid (cake) stream containing mostly grit/germ/fiber, is sent to the first dewater milling step 43 to break the solid particles (germ/grit/fiber), such that the starch and oil from the grit/germ/fiber solid particles are released. After milling at the Step 43, the solid is mixed with the liquid from a third solid/liquid separation step 49 and sent to a holding tank step 46. The back-set accounts for less than half of the cook water volume, so the solid (germ/grit/fiber) is able to stay in the same sized holding tank for more than double the holding time and at much lower Brix. This feature allows the grit/germ/fiber/starch to be quickly and easily soften/broken up, the starch to be liquefied, and oil to be released from the germ particles.

After the holding tank step 46, the slurry is sent to the second solid/liquid separation step 44 to dewater. The liquid separated from step 44 is recycled back to the slurry tank step 41. The cake from the second solid/liquid separation step 44 goes to a second dewater milling step 45. Subsequently, the cake is mixed with back-set water before the third solid/liquid separator step 49. The liquid from the third solid liquid separation step 49 is sent to the holding tank step 46. The cake from the solid liquid separation step 49 is sent to the fermenter for a fermentation step 23.

The liquid from the first solid/liquid separation at the step 42 contains most of the oil in the front end and is sent to a front end oil recovery system. A three phase nozzle centrifuge is normally used to separate the oil/emulsion/small germ particle from the liquefied starch solution at oil separation step 47. The light phase that contains most oil/emulsion/germ particles with small amount of liquefied starch solution is sent to another, smaller three phase separation centrifuge (decanter or disc centrifuge) to polish oil if needed. The heavy phase and underflow/cake phase from both three phase nozzle centrifuge step 47 and third solid liquid separate are sent to the fermentation step 23 to be first converted to a sugar then to an alcohol. The “beer” from the fermenter that contains about 15% to 17% alcohol goes to distillation step 24 for alcohol recovery. The resulting whole stillage devoid of alcohol from the bottom of distillation step 24 has an option to go to the first stage evaporator for pre-concentration from a normal 12 to 14% DS to 15 to 25% DS concentration, then followed by a germ cyclone to float any larger germs that are still in the whole stillage.

The use of the germ cyclone is able to increase the oil yield about 0 to 0.2 lb./Bu depending on the front grind system and the density of the concentrated whole stillage and the effectiveness of the germ cyclone operation. The de-germ fiber stream discharged from the bottom of the germ cyclone or the whole stillage discharged from the bottom of the distiller at step 24 is sent to a decanter centrifuge at the fiber separation step 25 to recovery fiber as wet DDG cake. The liquid recovered from the decanter is split into two streams: about 30% to 60% or more of the flow is used as a back-set (e.g., sending to step 49) and remaining 40% to 70% of the flow is sent to the evaporator step 27 to be concentrated to about 45% DS as a syrup byproduct.

The oil recovered at the front end system provides a much lighter color and lower fatty acid (about 5 to 9%) than similar oil recovered from the back-end of the process. The oil yield at the front end is affected by the grind size of the grain particles in the initial grinding step, the number of dewater milling stage at the front end, and the post-distillation hydroclone germ recovery efficiency. With one dewater milling system, the front-end oil yield is about 0.2 to 0.4 lbs/Bu. With two dewater milling stages in series, the front-end oil yield is about 0.3 to 0.5 lbs/Bu. With an additional de-germ system in the back end, the front-end oil yield is about 0.5 to 0.6 lbs/Bu. Not all of the oil present in the germ is able to be obtained at the front end oil recovery system. This is because the oil in the germ particles can only release less than half of the oil in the front end steps given the relatively short contact time of the water with the germ. More oil is released from the germ particles that can be recovered at the back end than at the front end of the process, because the long contact time in fermentation encourages oil leaching coupled with the presence of alcohol during fermentation, which can act as a solvent to extract more oil out during the fermentation step 23, distillation step 24, and/or in the evaporation step 25. Also more than half (60% to 70%) of the liquid from the centrifuge during the DDG cake recovery goes to the evaporator step 25, so that the oil in this stream cannot be recovered at the front end.

An additional back end oil recovery system step 26 is needed if higher oil yield is needed. In addition, if the corn used is old or are dried in a high temperature environment, the germ particle softening process becomes very slow. In such a case, more enzymes and larger holding tanks or decreased throughput (to give longer holding time to soften germ) are needed.

The above described improvement on the dry mill processes for producing valuable byproducts (like oil, protein and cellulose for secondary alcohol production etc.) has became a more efficient process than a typical wet mill process for ethanol production. However, many bio tech processes, such as four carbon alcohols (iso- and normal butanol) processes, still prefer to use pure starch/sugar from a wet mill process as a feedstock, because the four carbon alcohol processes need to use a sterilized sugar solution to minimize secondary bacterial metabolites. Also, in-situ alcohol recovery methods are used in order for the toxicity of carbon four alcohol to be avoided. These recovery techniques can be complicated by the presence of non-starch grain particles.

FIG. 5 illustrates a typical corn kernel structure 500 with two types of endosperm, floury endosperm 502 (soft/lose starch granules within a very thin protein matrix cell wall) and horny endosperm 504 (hard/tough starch granules in a very thick protein matrix cell wall).

In a wet mill process, many complex and costly operation steps are needed to separate/purified the starch from the protein of the horny endosperm. On the other hand, the starch in the protein matrix inside the horny endosperm can be easily converted to a liquefied starch in a liquefaction step in a dry mill process. However, this liquefied starch contains protein, oil, and other soluble solids which are not an ideal feedstock for many biotech processes.

### SUMMARY OF THE INVENTION

In some embodiments, the present invention separates the floury endosperm from horny endosperm using a corn dry mill process. In some other embodiments, the present invention separates the floury endosperm from horny endosperm using a corn wet mill process. In some embodiments, pure raw starch or liquefied starch are first produced mainly from the starch inside the floury endosperm. The pure raw starch can be used as a feedstock for bio-tech industry. In some other embodiments, the horny endosperm (including grit) along with all non-starch materials inside the corn kernel (such as, germs, fibers and soluble solids) are combined to produce ethanol and valuable byproducts (such as, protein, oil and cellulose) in a dry mill process.

In the following, optimization of a starch separation processes (e.g., separating the floury endosperm from the horny endosperm) and a process of purifying the starch are disclosed. The following four processes (a system 60 of FIG. 6, a system 70 of FIG. 7, a system 80 of FIG. 8, and a system 90 of FIG. 9) are selected embodiments of optimizing the starch separation and purification.

FIG. 6 illustrates a dry mill starch recovery system 60 in accordance with some embodiments of the present invention. In some embodiments, the system 60 comprises a starch recovering/isolation unit 60A including a liquefied starch separation step 61 and liquefied starch purification step 62. In some embodiments, the liquefied starch separation step 61 and the liquefied starch purification step 62 can be added on/or combined with a typical dry mill process. In some embodiments, the starch recovering unit 60A produces pure liquefied starch. The system 60 can be used to produce solid free liquefied starch (such as, 90%-100% pure, 95%-100% pure, or 99%-100% pure, which can be used for bio-tech processes), as well as ethanol, and valuable byproducts (protein and oil).

In some embodiments, the process 60 can begin with a milling step 21 using a hammer mill, roller mill, or other suitable dry grain grinding process. In the liquefaction step 22, the starch in the floury endosperm is able to be liquefied and the starch in the horny endosperm is still inside the protein matrix (bonding with protein as grit). The liquefied stream in the liquefaction step 22 (containing liquefied starch with all the solids such as germ, grit, fiber and soluble solid) are sent to a separation device (such as a paddle screen) to remove those solids in the liquefied starch separation step 61.

In the liquefied starch purification step 62, the liquefied starch from the step 61 can be further purified by filtration or using a centrifugation device to remove any fine solids. At the step 61, the rest of solids (grit, germ, and fiber) are sent to a grinding step 63 for further breaking up the interactions between the 1) starch and protein, 2) the starch and fiber and, 3) the starch and germ, such that the bonded starch can be free up and liquefied before sending to a fermentation step 23 and distillation step 24 for ethanol production. The system produces ethanol and value byproducts such as oil, protein and cellulose.

FIG. 7 illustrate a dry mill starch recovery system 70 in accordance with some embodiments of the present invention. The process 70 comprises a starch recovering/isolation unit 70A having a digestion step 71, a starch recovery/separation step 72, and a starch purification step 73 in a front end process (before fermentation) of a dry mill process.

At a milling step 21 in the system 70, the corn is first milled in a milling device, such as a hammer mill. At a step 71, after the milling step 21, the corn flour is sent to a digester along with an amount of process water (such as from a starch purification step 73). The pH of the solution/slurry at the step 71 is adjusted to have a pH around 7 to 9 and the temperature is kept just below the starch gelatinization temperature (around 50° C.), such that the starch inside the floury endosperm and a significant fraction of the horny endosperm can be freed/separated from the rest of the grain material.

At a starch recovery and separation step 72, freed starch is separated from larger particle size grit, germ, and fiber by using a screen device (such as a pressure screen or a paddle screen). The starch slurry from separation step 72 is sent to a starch purification device at a starch purifying step 73. The starch purification device can be cyclones or centrifuges, such that non-starch solids (oil, protein, germ, coarse fiber, fine fiber and soluble solid) can be removed. This purified starch at step 73 can be used as feedstock for some predetermined biotech processes, such as making butanol.

The larger size solids (such as germ, grit, and fiber) at the starch recovery/separation step 72 is sent to a liquefaction step 22, to produce ethanol and valuable byproducts such as oil, protein, and cellulose.

FIG. 8 illustrates a wet mill process starch recovering system 80 in accordance with some embodiments of the present invention. In some embodiments, the process 80 comprises a starch recovering/isolating unit 80A before the fermentation step 23. In some embodiments, the process 80 comprises a corn kernel softening step 11 (such as steeping), a grind milling step 12 for cracking open the steeped corn kernel, and a germ separating step 13.

At the germ separating step 13, the de-germ stream goes to a starch separating/recovering step 81, such that the “freed” starch can be separated from the “bound” starch and other non-starch material. Freed starch includes starch granules that are loose and largely free from attachment to other materials. Freed starch granules are often less than 35 uM in diameter. Bound starch includes starch granules that are physically attached to proteins, fibers, germ or combinations of these components.

At the starch separating/recovering step 81, the “freed” starch stream is sent to a starch purifying step 82 to produce purified starch as a feedstock for predetermined bio tech processes. The “bound” starch and non-starch material stream at the starch separation/recovery step 81(such as fiber, germ, and grit) are used to produce ethanol and valuable byproducts (such as oil, protein, and cellulose). In some embodiments, the bound starch and non-starch material stream can be sent to a liquefaction/scarification step 22. The system 80 produces ethanol and high value byproducts such as oil/germ, protein, and cellulose.

FIG. 9 illustrates a dry mill starch recovery system 90 in accordance with some embodiments of the present invention, which is similar to the system 70 of FIG. 7 with an additional sieving step.

In an aspect, A dry milling starch recovering method comprises, before fermenting, separating a first starch from a second starch, wherein the first starch comes from a floury endosperm and the second starch is inside a horny endosperm, sending the first starch to a starch purification device, and purifying the first starch forming a purified liquefied starch.

In some embodiments, the method further comprises providing the purified liquefied starch as a biotech feedstock. In other embodiments, the separating comprising forming a liquid phase containing a liquefied starch and a solid containing phase having starch bound with germ, grit, and fiber. In some other embodiments, the method further comprises grinding the solid containing phase to free the second starch from the horny endosperm. In some embodiments, the solid containing phase is sent to a fermenter. In other embodiments, the method further comprises generating alcohol using the solid containing phase. In some other embodiments, the method further comprises liquefying before the separating, such that the first starch is separated from the floury endosperm. In some embodiments, the horny endosperm comprises protein bound with the second starch contained inside the horny endosperm. In other embodiments, the purifying comprises removing oil and protein from the first starch.

In another aspect, a dry milling starch recovering method comprises subjecting a milled corn flour to a caustic chemical in a digester, adjusting a pH value of a solution containing the milled corn flour in the digester to a range of 7.5-9, maintaining a temperature of the solution in the digester below a starch gelatinization temperature, isolating an amount of freed starch from a remaining bound starch, and sending the bound starch for fermenting. In some embodiments, the caustic chemical comprises NaOH and Na₂CO₃. In other embodiments, the caustic chemical further comprises Na₂SO₃. In some other embodiments, the method further comprises grinding after the digester. In some embodiments, the method further comprises sending a starch slurry to purifying the freed starch after the isolating the amount of freed starch. In other embodiments, the method further comprises removing oil and fiber at the purifying the freed starch. In some other embodiments, the bound starch for fermenting comprises grit, germ, and fiber.

In another aspect, a wet mill starch recovering method comprises soaking or steeping an amount of corns, wet milling the corns to generate a free starch portion and a bound starch portion, separating the free starch portion and the bound starch portion, sending the free starch portion for starch purifying to produce purified starch, and sending the bound starch portion for fermenting. In some embodiments, the method further comprises removing germs before the separating. In other embodiments, the method further comprises liquefying before fermenting. In some other embodiments, the bound starch portion comprises fiber and grit. In some embodiments, the method further comprises producing alcohol using the bound starch portion. In other embodiments, the method further comprises producing butanol using the purified starch.

In another aspect, a dry milling starch recovering method comprises milling an amount of corn forming flour, separating the flour into a coarse flour portion and a fine flour portion, sending the fine flour portion to a caustic chemical in a digester to produce free starch, adjusting a pH value of a solution in the digester to a range of 7.5-9, maintaining a temperature of the solution in the digester below a starch gelatinization temperature, recovering the free starch, purifying the free starch to form purified starch, and sending the coarse flour portion for fermenting. In some embodiments, the method further comprises grinding between the recovering and the digester. In other embodiments, the method further comprises generating butanol using the purified starch.

### BRIEF DESCRIPTION OF THE DRAWINGS

The accompanying drawings, which are incorporated in and constitute a part of this specification, illustrate embodiments of the invention and, together with a general description of the invention given above, and the detailed description of the embodiments given below, serve to explain the principles of the invention.

FIG. 1 is a flow diagram of a typical wet-milling process and system for producing ethanol, protein meal and protein feed;

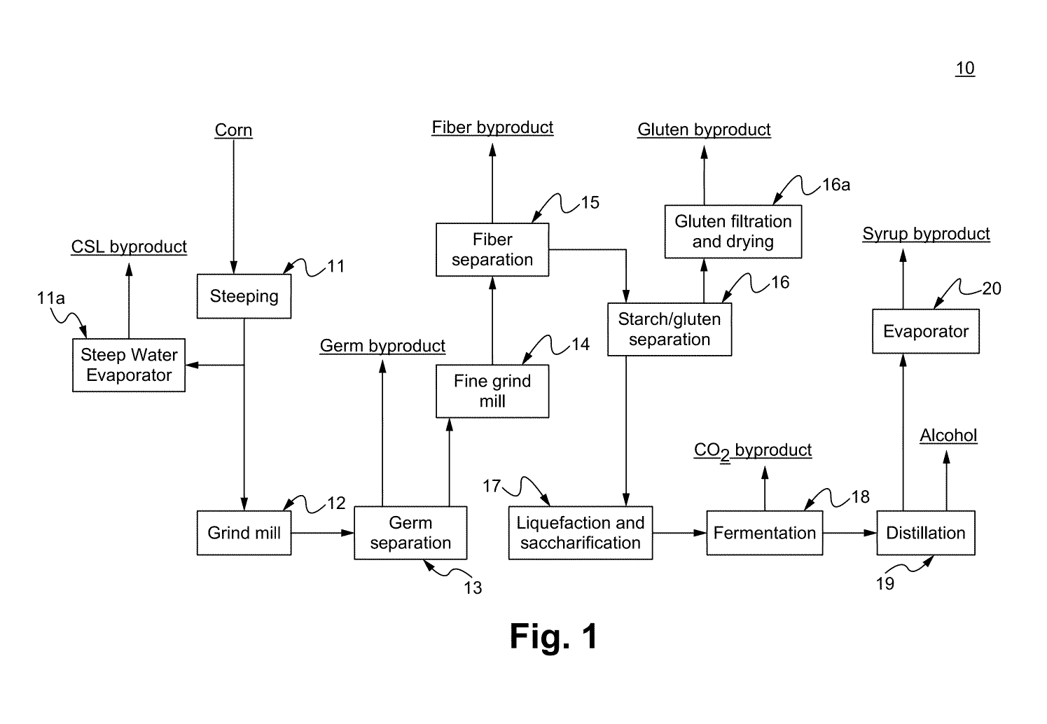

FIG. 2 is a flow diagram of a typical dry-milling process and system for producing ethanol and recovering oil and WDG in a back end process.

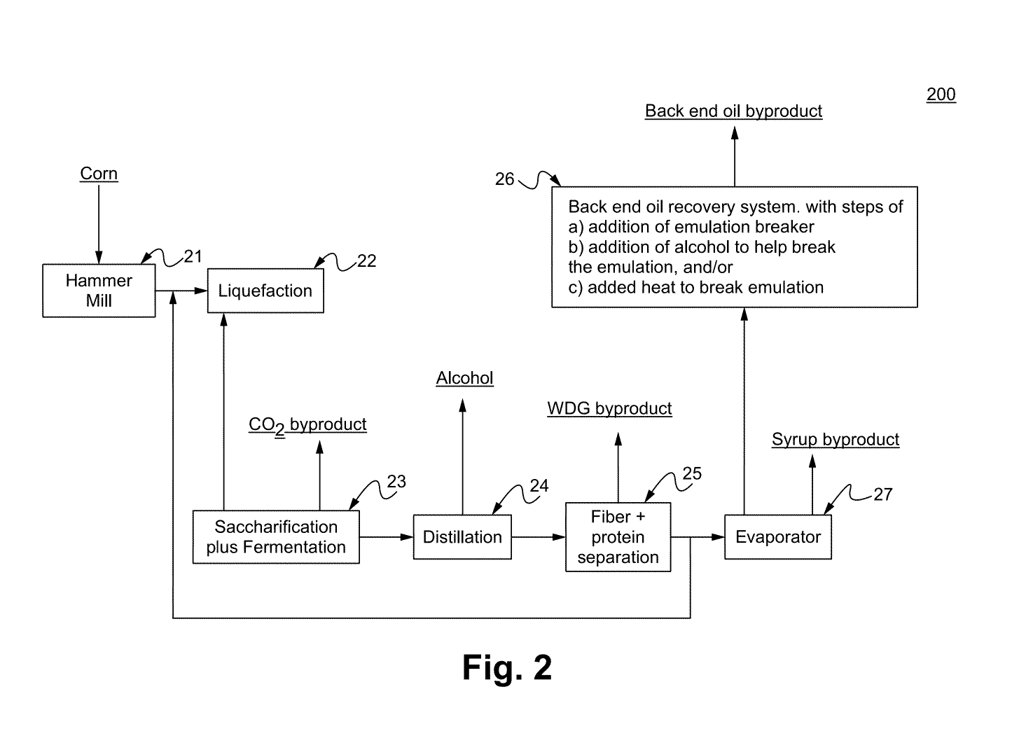

FIG. 3 is a flow diagram of a typical dry-milling process and system for producing ethanol and recovering oil and protein and WDG in a back end process.

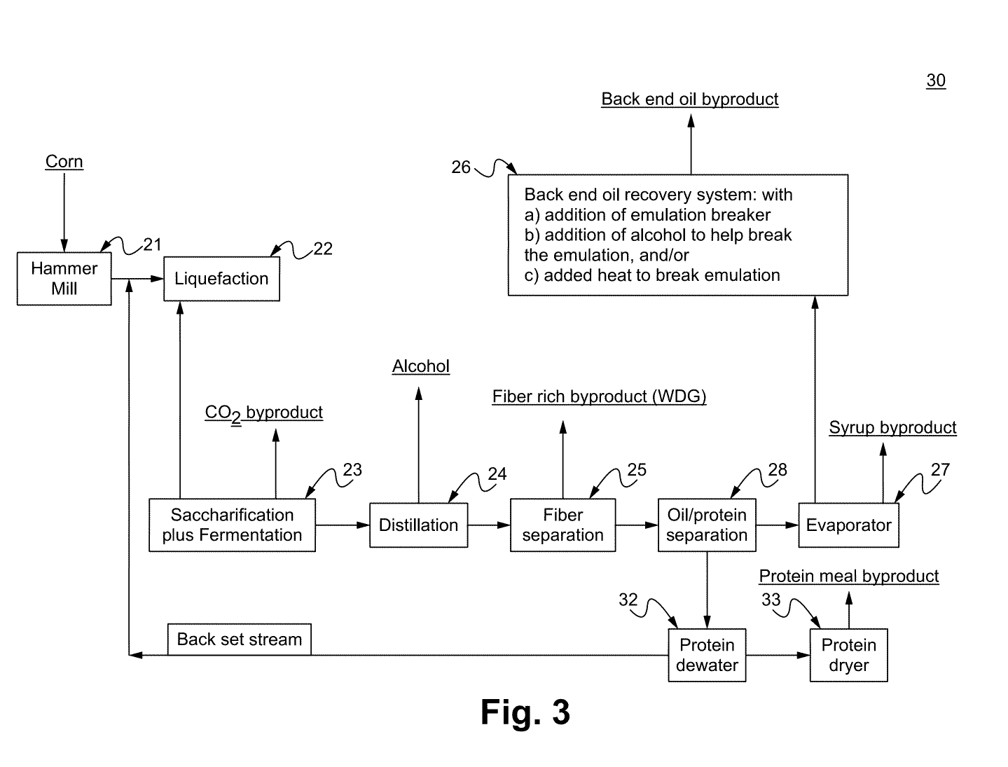

FIG. 4 is a flow diagram of a typical method and system for a dry mill process.

FIG. 5 illustrates a typical corn kernel structure.

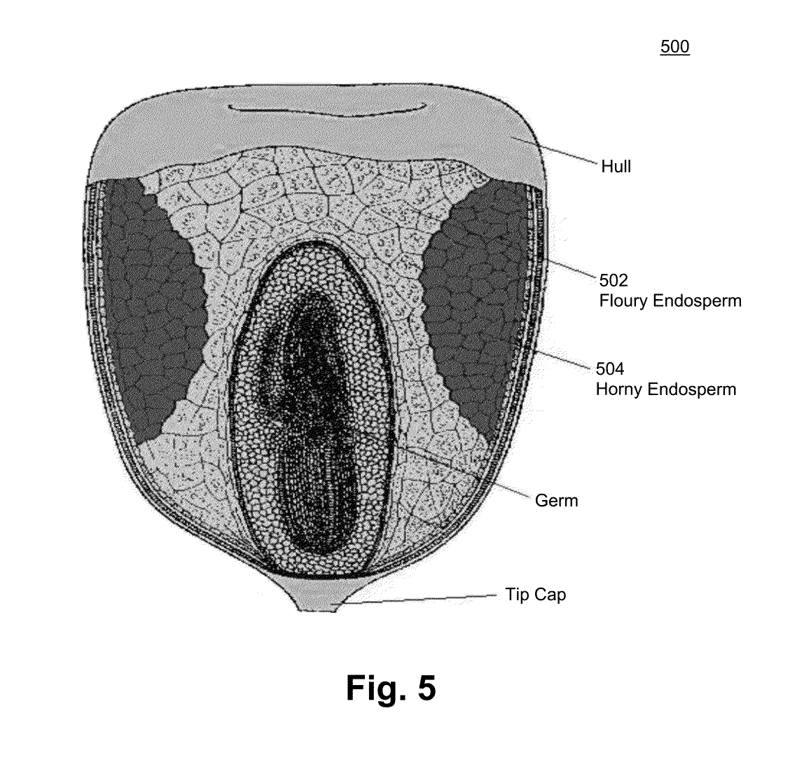

FIG. 6 illustrates a dry mill starch recovery system in accordance with some embodiments of the present invention.

FIG. 7 illustrates another dry mill starch recovery system in accordance with some embodiments of the present invention.

FIG. 8 illustrates a wet mill process starch recovering system in accordance with some embodiments of the present invention.

FIG. 9 illustrates a dry mill starch producing process in accordance with some embodiments of the present invention.

### DETAILED DESCRIPTION OF THE PREFERRED EMBODIMENT

The majority of the starch in the corn kernel are inside the two types of endosperm: floury endosperm (soft endosperm) and horny endosperm (hard endosperm; commonly called “grit”). The starch granules inside floury endosperm can be easily separated/removed resulting in purified starch. However, the starch granules inside the horny endosperm are protected by a strong protein matrix, which is difficult to be separated from the starch granule to produce purified starch.

The corn wet milling process is complex and costly, which is mainly aimed to produce as much pure starch as possible from both endosperms. Although the dry milling process can easily convert the starch in floury and horny endosperm to sugar then to alcohol and produce high value byproducts such as oil, protein, and cellulose, the typical dry mill plant cannot produce purified sugar or raw starch as feedstock for renewable energy and biotech processes.

In some embodiments, the present invention separates/isolates the starch in floury endosperm from the rest of substances inside the corn first and produces purified raw starch or liquefied starch as a feedstock for the use in renewable energy and biotech technology, such as making butanol, which favors purified starch.

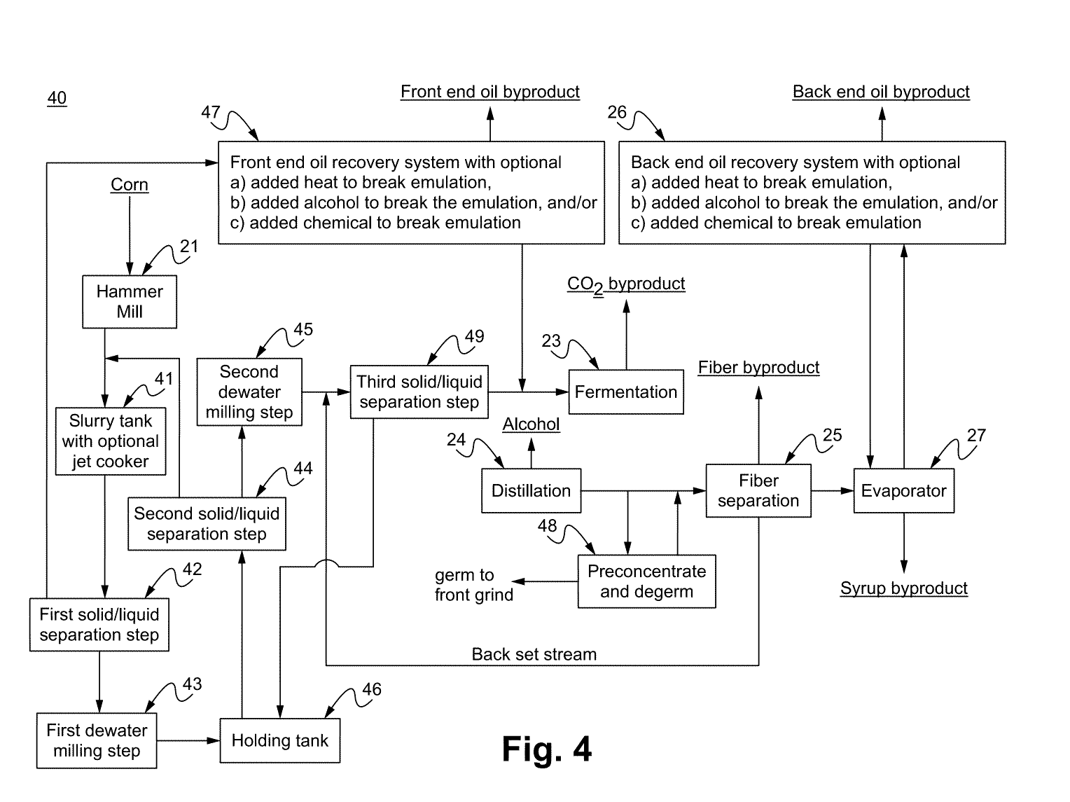

Next, in some embodiments, the rest of substances (such as germ, starch bond with protein and fiber, and others) inside the corn kernel is sent to one of the dry mill processes (such as process 40 in the FIG. 4) to produce ethanol and valuable byproducts such as oil, protein and cellulose.

The following three flow diagrams illustrate several embodiments to produce the raw starch/liquefied starch that are needed for renewable energy and biotech processes.

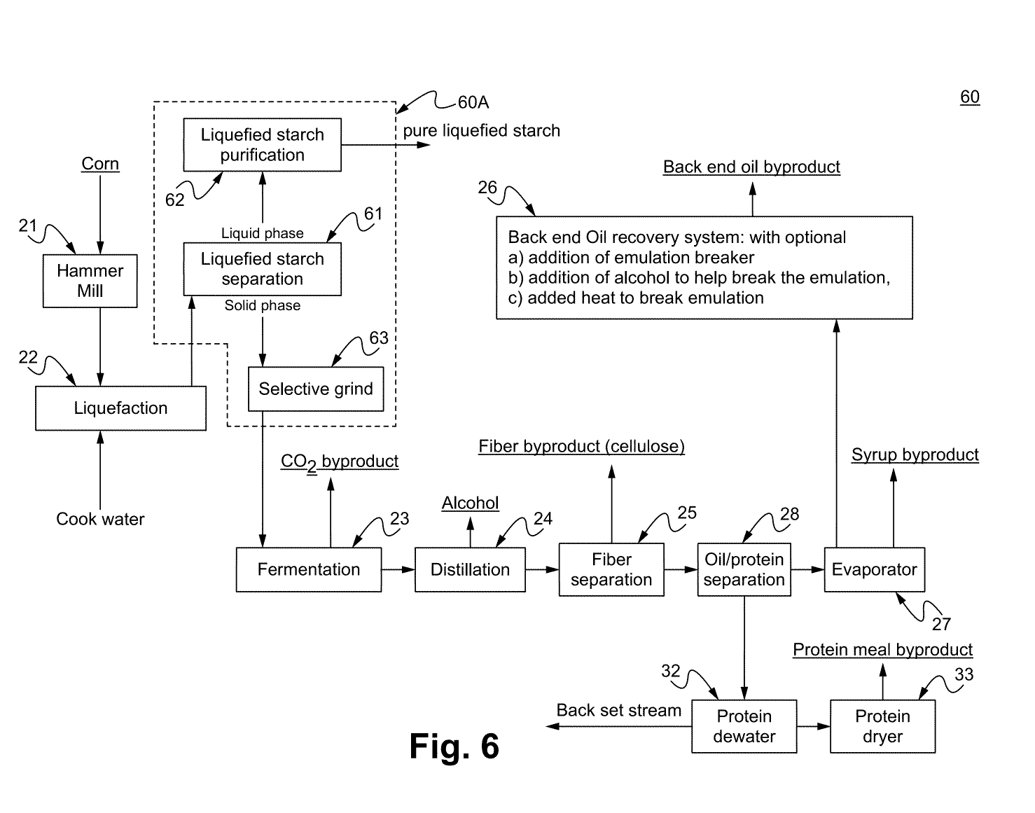

FIG. 6 illustrates a dry mill starch recovery/isolation system 60 in accordance with some embodiments of the present invention. In some embodiments, the starch recovering unit 60A produces pure liquefied starch. In some embodiments, the process 60 comprises a liquefied starch separation step 61, a liquefied starch purification step 62, and a selective grinding step 63. The process can be used together or added onto a typical dry milling process.

At a milling step 21, the corn is fed to a hammer mill, roller mill, or other suitable dry grain grinding mill to produce corn flour with a predetermined particle size distribution by selecting an appropriate screen size. At a liquefaction step 22, cook water and enzyme are added to the corn flour. The liquefaction step 22 includes using a slurry tank, a jet cooker, a selective grinding device, a holding tank, and a fiber separation device, which occurs before a fermentation step 23.

At a liquefied starch separation step 61, the liquefied starch from the liquefaction step 22 is separated from the rest of the material. Any screen separation devices (such as a pressure screen, a paddle screen, or a combination thereof) can be used at the step 61.

The liquid portion from the step 61 contains mainly liquefied starch with small amount of oil, protein, and soluble solid, which are sent to a liquefied starch purification step 62, such that the oil and protein are removed by using a filtration device, such as a vacuum drum or a centrifugal device (such as nozzle centrifuge or new disc decanter). The purified liquefied starch from the liquefied starch purification step 61 is able to be used as a feedstock for biotech processes.

In some embodiments, the solid phase from the liquefaction separation step 61 is sent to a selective milling step 63 to further free and liquefy the “bound starch.” Then, the output from the selective milling step 63 is sent to one of the improved dry milling processes (e.g., process 40 in FIG. 4) such as fermentation step 23 to produce ethanol and valuable byproducts (such as oil, protein, and cellulose/DDGS).

The pure liquefied starch from the liquefied starch purification step 62 has a sugar content of 10 to 40% DS (dry solids) and a protein content between 0.3% to 3% DS. The sugar and protein contents are variable depending on the system setup and operational conditions employed. The pure liquefied starch yield can vary depending on the types of corn used, and operation conditions and equipment used. In general, a higher yield starch normally produces a lower purity liquefied starch. In some embodiments, the starch yield ranges from 30% to 85% of the starch in corn.

In the following, several operations conditions are selected to be controlled based on preselected yield and purity targets, including a) choosing #2 yellow corns resulting in a higher yield and a high purity of liquefied starch than choosing waxy corn, b) using a small screen opening on a hammer mill or roller mill resulting in a high yield but lower purity, c) using a larger screen opening on liquefied starch separation step resulting in a higher yield but lower purity, d) using a lower liquefied temperature in the liquefaction step resulting in a lower yield but higher purity, e) using a longer holding time in the liquefied step resulting in a higher yield but a lower purity, f) using a lower degree of liquefaction by choosing the type and dosage of enzyme, which can be controlled to provide a lower yield but higher purity, g) using a smaller pore size opening on a vacuum drum filter cloth resulting in a high purity, and h) using a higher G force on a centrifuge resulting in a higher purity.

The liquefied starch produced using the process 60 contains non-starch soluble minerals and vitamins from corn plus some amount of soluble and insoluble protein and oil.

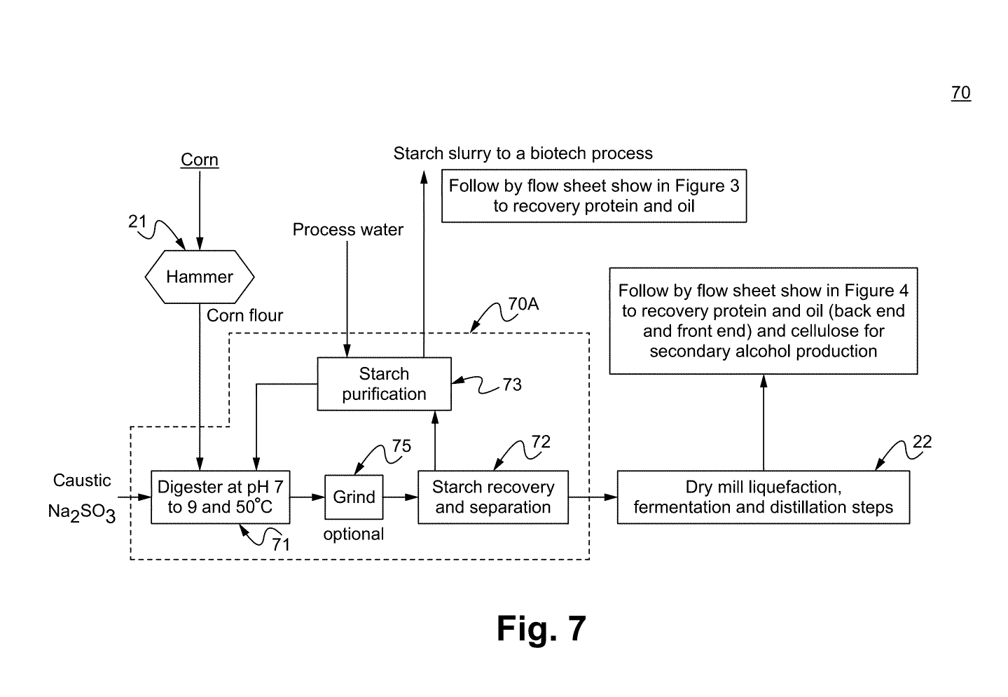

FIG. 7 illustrates a dry mill starch recovery system 70 in accordance with some embodiments of the present invention. In the process 70, corns go through a hammer mill or roller mill at a milling step 21 to produce a predetermined particle size distribution corn flour by selecting the screen size and airflow rate.

At a digesting step 71, corn flour is added to a digestion tank with an amount of process water to have a concentration of 10 to 40% DS. The pH of the slurry is adjusted to 7-9 with a caustic chemical (e.g., NaOH), soda ash (Na₂CO₃) or lime substances. In some embodiments, an amount of grit softening agent (such as Na₂SO₃) is also added to the digestion process. The temperature at the digester is maintained just below the starch gelatinizing temperature (around 50° C. to 55° C.) for 10 min to 2 hour at the digestion step 71.

By using the digesting step 71, the protein matrix cell wall in the endosperm is broken down by the caustic substances and the starch granules are released. All or substantially all of the starch in the floury endosperm and a portion of the starch in the horny endosperm are “freed” from the corn kernel structure in the digestion step 71.

In some embodiments after the digestion step 71, a grind step 75 is able to be used to increase the starch yield. The substance in the digestion step 71 or combine of step 71 and 75 is sent to a starch recovery/separation/isolation step 72. At the step 72, a screen type separator is used (such as pressure screen, paddle screen, a combination thereof, and other types of screen separator) to separate the freed starch from the other larger insoluble corn particles. The screen opening size can be ranging from 45 micron to 250 micron depending on the yield and purity of the starch desired.

At the step 72, a stream that contains mainly “freed” starch is sent to a starch purification step 73, such that all the non-starch material (such as oil, protein (soluble and insoluble) and soluble solid inside the corn kernel) are washed/removed.

At the step 73, multi-stage hydrocyclones or disc stack nozzle centrifuge with counter current washing set up are often used. The purified starch slurry after the step 73 can have a 35% to 40% DS concentration with a low protein content (0.2 to 2% in dry base).

The other stream from the starch recovery/separation step 72 that contains mainly the bound starch and all other non-starch material from the corn kernel are sent to a dry mill liquefaction step 22 for producing ethanol and valuable byproducts by using a dry mill processes. In some embodiments, the process 70 has a pure raw starch yield from 30 to 50% of the starch inside the corn kernel.

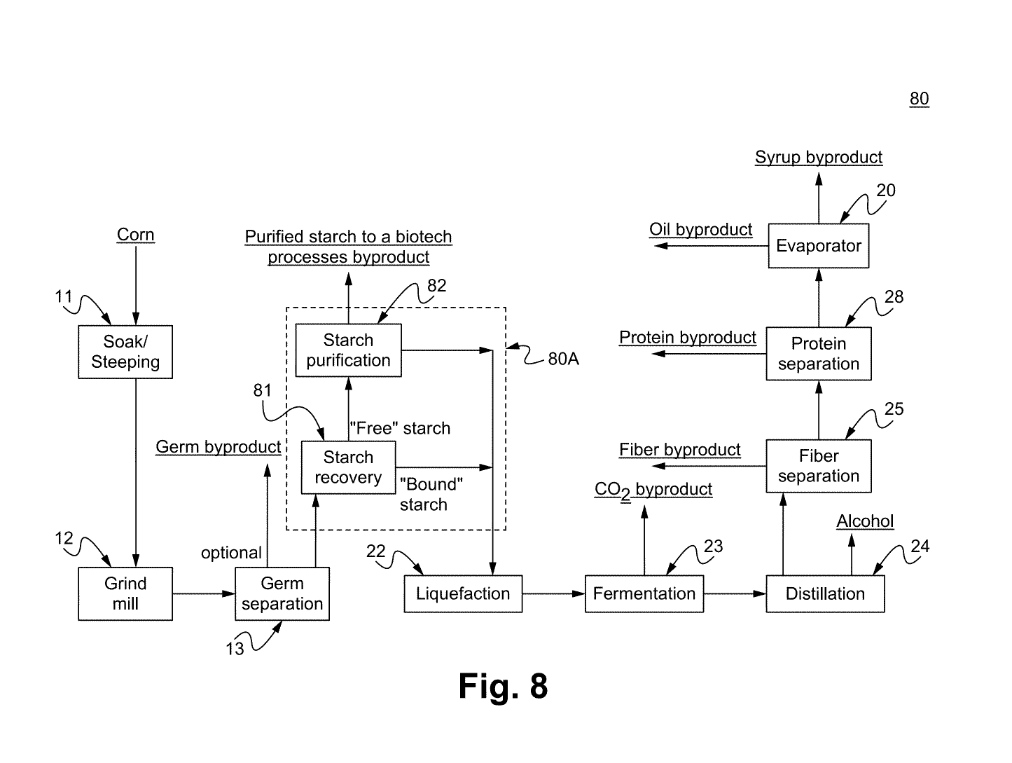

FIG. 8 illustrates a wet mill process starch recovering system 80 in accordance with some embodiments of the present invention. In some embodiments, the process 80 is able to have a high pure starch yield and recover germs for producing food grade corn oil instead of lower quality industrial grade corn oil.

In the process 80, the corn goes through a soak/steeping step 11. In some embodiments, a grind milling step and a germ separation step 13 are performed to separate and recover germs. In some embodiments, the de-germ stream at the germ separation step 13 is sent to a fine grinding device (such as, for example a disc mill) to further break the bonds between starch and protein matrix, which is then sent to a starch recovery step 81, such that “freed starch” is separated and recovered from “bound starch” and non-starch material inside the corn kernel.

The starch stream from step 81 is sent to a starch purification step 82 to wash/purify the starch. The washed, purified starch slurry (35 to 40% DS) from the starch purification step 82 is used as a feedstock for predetermined biotech processes, such as making butanol. The bound starch and non-starch material from the corn kernel from step 81 and 82 are sent to a liquefaction step 22 and fermentation step 23 to produce ethanol and byproducts including oils, proteins, and fiber/DDGS. In some embodiments, pressure screens and paddle screens are used at step 81 and multi-stage cyclone or disc stack nozzle centrifuge are used at step 82 with counter current washing set up alone or in various working combinations.

The process 80 provides a higher starch yield and a high purity with an option to recover germ for producing a food grade corn oil.

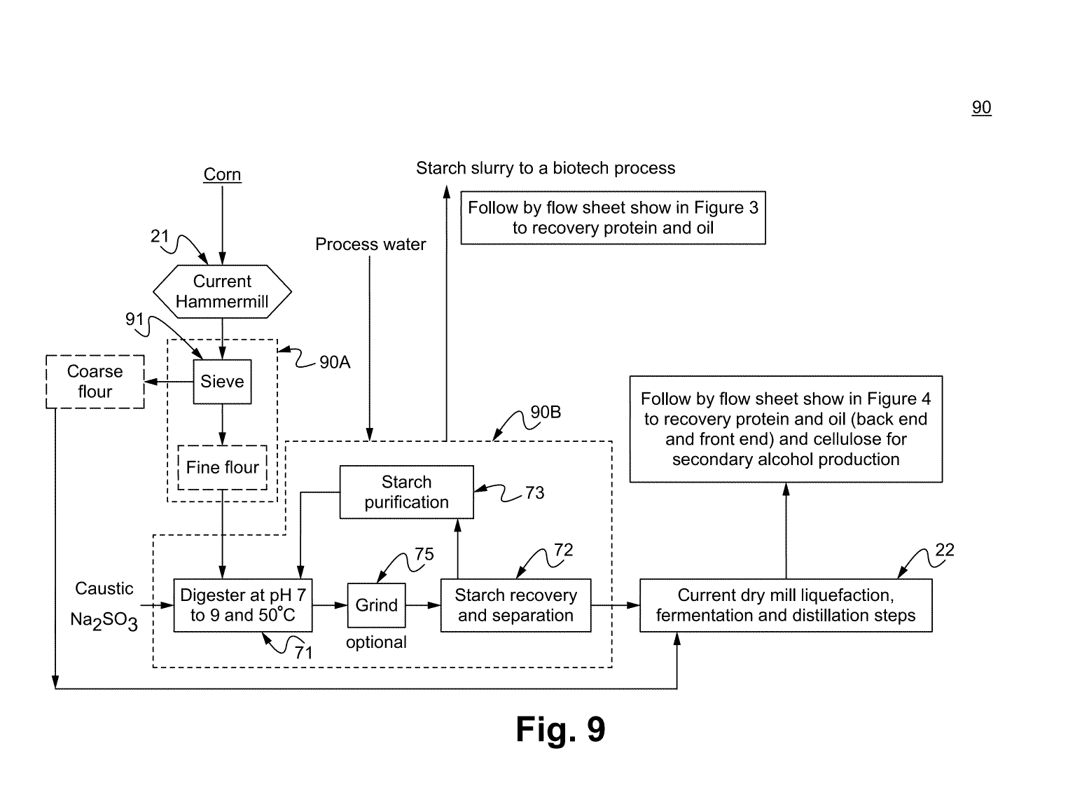

FIG. 9 illustrates a dry mill starch producing process 90 in accordance with some embodiments of the present invention. In some embodiments, the process 90 comprises a process 90B similar to the process 70 of FIG. 7. In some embodiments, the process 90 comprises the process 90B and a sieving process 90A.

In some embodiments, the sieving step 91 is introduced to separate hammer milled or roller milled corn into a coarsely ground corn fraction and a finely ground corn fraction. The coarsely ground corn has a higher concentration of fiber and horny endosperm. The finely ground corn has a higher concentration of floury endosperm. By diverting the fraction with higher flourly endosperm content, the amount of basic solution that is required to bring a given amount of material to pH 9 is reduced because of the lower buffering capacity of the floury endosperm when it is compared with the horny endosperm

While the present invention has been illustrated by a description of various embodiments and while these embodiments have been described in considerable detail, it is not the intention of the applicant to restrict or in any way limit the scope of the appended claims to such detail. For example the process described herein can use a sugar solution coming from other bio-processes, such as sugar cane and five carbon sugar from other cellulose raw materials. For another example, although various systems and methods described herein have focused on corn, virtually any type of grain, including, but not limited to, wheat, barley, sorghum, rye, rice, oats and the like, can be used. Using the processes described herein, purified (white) grain fiber can be produced to be used in a paper industry or as a feedstock for secondary alcohol production. Further, using the processes described herein, purified sugar solution can be produced to be used in green technology, existing food production processes (such as, for example, citric acid and lysine), and biotech processes. Thus, the invention in its broader aspects is therefore not limited to the specific details, representative apparatus and method, and illustrative example shown and described. Accordingly, departures can be made from such details without departing from the spirit or scope of applicant's general inventive concept.

Experiments

### EXAMPLE 1

Milled corn was obtained from a commercially operating ethanol plant using hammer mills equipped with 7/64″ screens. The milled corn was sent through a #35 USA standard testing sieve (500 _{M}M). 62% of the flour passed through the screen and 38% was captured on the screen. The material which passed through the screen (sub 500 micron) was collected and used for the following corn starch extraction test.

Seventy five (75) grams of the sub 500 micron milled corn was added to 500 mLs of water and thoroughly mixed. Sodium carbonate (soda ash) was added to the mixture until a pH of 9 was reached. The corn slurry was placed in a lab Waring blender and blended for 2 minutes. The blended corn slurry mixture was then placed on a hot plate with magnetic stirrer and brought up to 50° C. and held at this temperature for 1 hour under constant agitation.

The heated corn slurry was then poured into two 300 mL containers and centrifuged in a lab centrifuge at 2000 rpm's for 1 minute. The supernatant was decanted off and discarded and the suspended solid pellet retained.

The corn pellet in each centrifuge tube was re-suspended with approximately 250 mLs of warm tap water by adding the water and shaking the container and then placed in the lab centrifuge for 1 minute at 2000 rpm. The supernatant was again decanted off and discarded and the suspended solid pellet retained.

The corn pellet in each centrifuge tube was re-suspended for a third time with approximately 250 ml of warm tap water by adding the water and shaking the container and then placed in the lab centrifuge for 1 minute at 2000 rpm. The supernatant was decanted off and discarded and the suspended solid pellet retained.

The corn pellet was re-suspended a fourth time with a small amount of water and poured over a #325 USA standard testing sieve (45 μM). Additional water was used to wash the corn particulate residing on the top of the filter screen to remove as much fine suspended material as practical. The corn particulate that did not go through the screen (fiber) was dried in a 50° C. convection lab oven overnight.

The corn material (starch) that did pass through the #325 USA standard testing sieve was put into a 300 mL centrifuge tube and spun on lab centrifuge at 2000 rpm's for 1 minute. The water was decanted and discarded and the solid pellet retained. Approximately 2000 ml of water was added to starch pellet to re-suspend. This suspension was allowed to stand for 45 minutes for the starch to settle to the bottom of the container. After 45 minutes settling time the supernatant water was decanted and discarded. The remaining corn pellet was dried overnight in at 50° C. in a lab convection oven.

The resulting washed and dried corn particles greater than 50 μM and less than 50 μM fractions were analyzed for protein content. The material greater than 50 μM was yellowish brown in color and contained 14.1% protein, dry matter basis. The material smaller than 50 μM was nearly pure white in color and contained 2% protein, dry matter basis.

To utilize, the present invention is able to recover starch, before fermentation, to be used as a biotech feedstock.

In operation, the present invention is able to recover starch before fermenting in a dry mill system and to recover free starch in a wet mill system. The starch recovered can be used as a feedstock for biotech industry, such as for making butanol. In the wet mill system, the portion contains the bound starch is sent to a fermentation process for producing alcohol. In an embodiment, a dry milling starch recovering method comprises before fermenting, separating a first starch from a second starch, wherein the first starch comes from a floury endosperm and the second starch is inside a horny endosperm, sending the first starch to a starch purification device, and, purifying the first starch forming a purified liquefied starch.

The present invention has been described in terms of specific embodiments incorporating details to facilitate the understanding of principles of construction and operation of the invention. Such reference herein to specific embodiments and details thereof is not intended to limit the scope of the claims appended hereto. It is readily apparent to one skilled in the art that other various modifications can be made in the embodiment chosen for illustration without departing from the spirit and scope of the invention as defined by the claims.

What is claimed:

1. A dry milling starch recovering method comprises:
a) before fermenting, separating a first starch from a second starch, wherein the first starch comes from a floury endosperm and the second starch is inside a horny endosperm;
b) sending the first starch to a starch purification device; and
c) purifying the first starch forming a purified liquefied starch.

2. The method of claim 1, further comprising providing the purified liquefied starch as a biotech feedstock.

3. The method of claim 1, wherein the separating comprising forming a liquid phase containing a liquefied starch and a solid containing phase having starch bound with germ, grit, and fiber.

4. The method of claim 1, further comprising grinding the solid containing phase to free the second starch from the horny endosperm.

5. The method of claim 4, wherein the solid containing phase is sent to a fermenter.

6. The method of claim 4, further comprising generating alcohol using the solid containing phase.

7. The method of claim 1, further comprising liquefying before the separating, such that the first starch is separated from the floury endosperm.

8. The method of claim 1, wherein the horny endosperm comprises protein bound with the second starch contained inside the horny endosperm.

9. The method of claim 1, wherein the purifying comprises removing oil and protein from the first starch.

10. A dry milling starch recovering method comprises:
a) subjecting a milled corn flour to a caustic chemical in a digester;
b) adjusting a pH value of a solution containing the milled corn flour in the digester to a range of 7.5-9;
c) maintaining a temperature of the solution in the digester below a starch gelatinization temperature;
d) isolating an amount of freed starch from a remaining bound starch; and
e) sending the bound starch for fermenting.

11. The method of claim 10, wherein the caustic chemical comprises NaOH and Na₂CO₃.

12. The method of claim 11, wherein the caustic chemical further comprises Na₂SO₃.

13. The method of claim 10, further comprising grinding after the digester.

14. The method of claim 10, further comprising sending a starch slurry to purifying the freed starch after the isolating the amount of freed starch.

15. The method of claim 14, further comprising removing oil and fiber at the purifying the freed starch.

16. The method of claim 10, wherein the bound starch for fermenting comprises grit, germ, and fiber.

17. A wet mill starch recovering method comprising:
a) soaking or steeping an amount of corns;
b) wet milling the corns to generate a free starch portion and a bound starch portion;
c) separating the free starch portion and the bound starch portion;
d) sending the free starch portion for starch purifying to produce purified starch; and
e) sending the bound starch portion for fermenting.

18. The method of claim 17, further comprising removing germs before the separating.

19. The method of claim 17, further comprising liquefying before fermenting.

20. The method of claim 17, wherein the bound starch portion comprises fiber and grit.

21. The method of claim 17, further comprising producing alcohol using the bound starch portion.

22. The method of claim 17, further comprising producing butanol using the purified starch.

23. A dry milling starch recovering method comprises:
a) milling an amount of corn forming flour;
b) separating the flour into a coarse flour portion and a fine flour portion;
a) sending the fine flour portion to a caustic chemical in a digester to produce free starch;
b) adjusting a pH value of a solution in the digester to a range of 7.5-9;
c) maintaining a temperature of the solution in the digester below a starch gelatinization temperature;
d) recovering the free starch;
d) purifying the free starch to form purified starch; and
e) sending the coarse flour portion for fermenting.

24. The method of claim 23, further comprising grinding between the recovering and the digester.

25. The method of claim 23, further comprising generating butanol using the purified starch.
